(19) United States
(12) Patent Application Publication
(10) Pub. No.: US 2005/0267562 A1
(43) Pub. Date: Dec. 1, 2005

(54) Stent-reduction sleeve

(75) Inventors: Ryan A. Jones, Higley, AZ (US); John D. Kantor, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/129,454

(22) Filed: May 13, 2005

Related U.S. Application Data
(60) Provisional application No. 60/570,914, filed on May 13, 2004.

(51) Int. Cl. A61F 2/06

(52) U.S. Cl. 623/1.11

(57) ABSTRACT

The present invention provides a method of reducing a stent. A stent framework is provided and inserted into a stent-reduction sleeve having a plurality of apertures formed therein. The stent-reduction sleeve is compressed towards a central axis of the stent framework with a substantially uniform inwardly directed radial force that is generated on the inserted stent framework. The stent diameter of the stent framework is reduced based on the amount of compression. A system for treating a vascular condition and a stent including a sleeve-compressed stent framework are also disclosed.

### RELATED APPLICATIONS

This application claims the benefit of U.S. Provisional Patent Application 60/570,914 filed May 13, 2004.

### FIELD OF THE INVENTION

This invention relates generally to biomedical stents. More specifically, the invention relates to methods of reducing the diameter of self-expanding or balloon-expandable stents during manufacture.

### BACKGROUND OF THE INVENTION

Endovascular stents and stent delivery assemblies are utilized in a number of medical procedures to restore the function of bodily lumens. With generally open cylindrical structures, the stents usually have apertured or lattice-like walls of a metallic or polymeric base, and can be either balloon expandable or self-expanding. In its expanded configuration, the stent supports and reinforces vessel walls while maintaining a vessel in an open, unobstructed condition.

Stents are conventionally one of two types, either inflation-expandable or self-expanding. Inflation-expandable stents are crimped to their reduced diameter about a balloon portion of a delivery catheter, introduced via the catheter into a lumen of a body vessel, maneuvered to the deployment site, and expanded to the vessel diameter by the balloon being inflated with inflation fluid or other expansion means. After the stent has been expanded to a larger diameter, the balloon is then deflated and removed, leaving the stent in place at the treatment site.

Self-expanding stents can be manufactured from expandable heat-sensitive materials that allow for phase transformation of the materials to occur at set temperatures. Self-expanding stents, which are often formed from super-elastic shape-memory metals such as nitinol and nickel-titanium alloys, expand from a compressed state when they are advanced out of the distal end of a delivery catheter into a body lumen. A delivery sheath of an elastic material may be used to keep a self-expanding stent compressed until it is ready for deployment, at which time the sheath is slipped off or otherwise removed. Any balloon-expandable or self-expanding stent that is delivered to a treatment site by a catheter needs to be secured and retained to that catheter to avoid proximal or distal translocation during handling and insertion. Additionally, the stent should be crimped in such a way as to avoid damage to the balloon or catheter, as well as distortion of the stent, thereby minimizing abrasion to vessel walls when the stent is deployed.

Various types of mechanisms have been used in crimping tools for compressing and securing a stent onto a delivery catheter: a collet with segmented jaws (U.S. Pat. No. 6,167,605), one or more moving or sliding plates (U.S. Pat. Nos. 6,092,273 and 6,481,262), segmental radial compression (U.S. Pat. No. 6,629,350), semi-circular shaped cams (U.S. Pat. No. 6,082,990), a flexible loop for radial compression (U.S. Pat. No. 6,063,102), a conical bore (U.S. Pat. No. 5,992,000), a piston and a compressible elastomeric sleeve (U.S. Pat. No. 6,009,614), a flexible Mylar sheet and rigid slotted panel (U.S. Pat. No. 6,141,855), an expandable bladder/elastic tube (U.S. Pat. No. 6,108,886), and a rubber tube and compression spring or a coiled filament (U.S. Pat. No. 6,240,615).

One of the difficulties with any of the aforementioned crimping devices is the possibility of non-uniform crimping forces being applied to the stent, which result in non-uniform crimps, and potential scoring, distortion, marking, or abrasion of the stent periphery. Crimping problems can be exacerbated by the small size of the stents, which are typically between one millimeter and three millimeters in diameter when rolled down and from about four millimeters to over thirty millimeters in length. An additional concern with existing stent-crimping equipment is that stent coatings can be damaged. Recent developments in drug-coated stents require improved handling during crimping or roll-down to avoid damaging any drug-polymer coatings and to prevent transfer of any unapproved materials into the coating.

Some stent crimping systems use sleeves or sheaths of polytetrafluoroethylene or other smooth materials placed around stents to help protect stent coatings and surfaces while the stents are being crimped or rolled down. Unfortunately, this sheath material may fold over on itself, abrading the stent coating, causing non-uniform compressive forces, and decreasing the effectiveness of the crimping technique.

The increased use of drug-polymer coatings on stents presents an even greater need for an improved crimping process that protects the stent coatings as well as the stent framework. Such an improved process would minimize damage to the coatings, thereby providing higher yields in manufacturing. The process should accommodate various coating formulations and stents of different diameters and lengths.

### SUMMARY OF THE INVENTION

One aspect of the invention provides a method of reducing a stent. The method includes providing a stent framework. The stent framework is inserted into a stent-reduction sleeve including a plurality of apertures formed therein. The stent-reduction sleeve is compressed towards a central axis of the stent framework. A substantially uniform inwardly directed radial force is generated on the inserted stent framework, and the stent diameter of the stent framework is reduced based on the compression.

Another aspect of the invention provides a system for treating a vascular condition. The vascular condition treatment system includes a stent-delivery catheter including a catheter body and a sleeve-compressed stent coupled to the catheter body. The sleeve-compressed stent includes a stent framework having a pre-deployment stent diameter that has been reduced by a stent-reduction sleeve. The stent-reduction sleeve includes a plurality of apertures formed therein to minimize folding of the stent-reduction sleeve when the stent framework is compressed.

Another aspect of the invention is a sleeve-compressed stent framework. The stent framework has a pre-deployment stent diameter that has been reduced by a stent-reduction sleeve. The stent-reduction sleeve includes a plurality of apertures formed therein to minimize folding of the stent-reduction sleeve during compression of the stent framework.

The present invention is illustrated by the accompanying drawings of various embodiments and the detailed description given below. The drawings should not be taken to limit the invention to the specific embodiments, but are for explanation and understanding. The detailed description and drawings are merely illustrative of the invention rather than limiting, the scope of the invention being defined by the appended claims and equivalents thereof. The foregoing aspects and other attendant advantages of the present invention will become more readily appreciated by the detailed description taken in conjunction with the accompanying drawings.

### BRIEF DESCRIPTION OF THE DRAWINGS

Various embodiments of the present invention are illustrated by the accompanying figures, wherein:

FIG. 1 illustrates a system for treating a vascular condition including a stent-delivery catheter and a sleeve-compressed stent coupled to the catheter, in accordance with one embodiment of the current invention;

FIG. 2 illustrates a stent-reduction sleeve with an array of diamond-shaped apertures, in accordance with one embodiment of the current invention;

FIG. 3 illustrates a stent-reduction sleeve with an array of longitudinally elongated slotted apertures, in accordance with one embodiment of the current invention;

FIG. 4 illustrates a stent-reduction sleeve with an array of segmented longitudinal slotted apertures, in accordance with another embodiment of the current invention;

FIG. 5 illustrates a stent-reduction sleeve with a set of perforations, in accordance with one embodiment of the current invention;

FIG. 6 illustrates a stent-reduction sleeve comprising a microporous material, in accordance with one embodiment of the current invention;

FIG. 7A, FIG. 7B, FIG. 7C and FIG. 7D show cross-sectional views of a method for reducing a stent, in accordance with one embodiment of the current invention; and

FIG. 8 is a flowchart of a method for reducing a stent, in accordance with one embodiment of the current invention.

### DETAILED DESCRIPTION OF THE PRESENTLY PREFERRED EMBODIMENTS

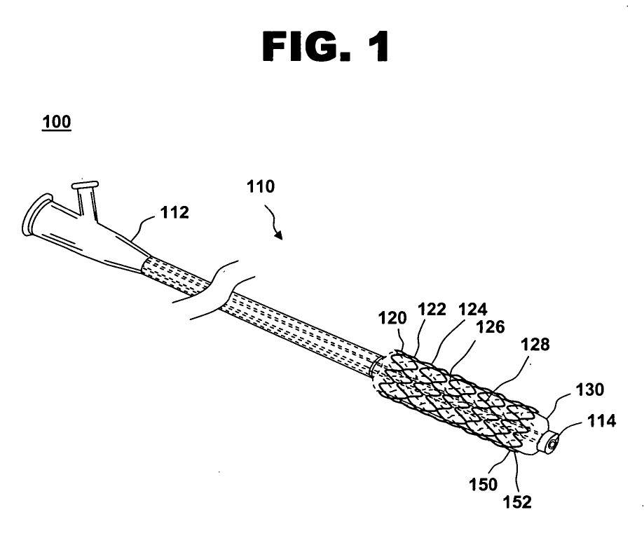

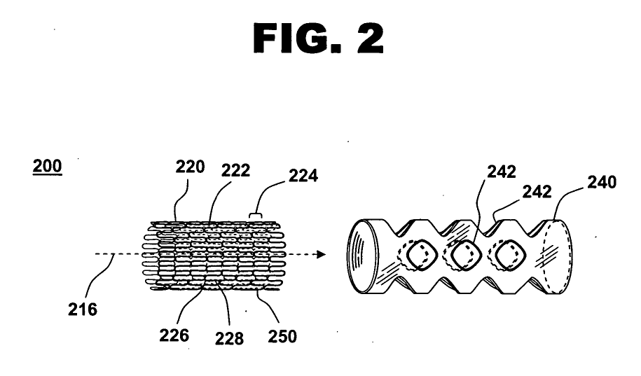

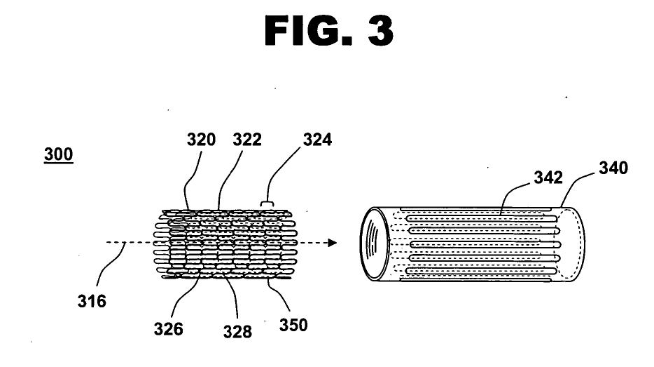

FIG. 1 illustrates a system for treating a vascular condition including a stent-delivery catheter and a sleeve-compressed stent coupled to the catheter, in accordance with one embodiment of the present invention at 100. Vascular condition treatment system 100 includes a stent-delivery catheter 110 and a sleeve-compressed stent 120. Stent-delivery catheter 110 includes a catheter body 112 with a guidewire lumen 114. Sleeve-compressed stent 120 includes a stent framework 122 that has been reduced by a stent-reduction sleeve to a pre-deployed stent diameter, the stent-reduction sleeve being described in conjunction with FIG. 2, and related embodiments in FIGS. 3, 4, 5, 6 and 7. The stent-reduction sleeve includes a plurality of apertures formed therein to minimize or eliminate the folding of the stent-reduction sleeve during compression of stent framework 122. Folding or buckling of non-apertured sleeves found in current art can occur, for example, when stents are reduced in diameter to fifty percent or less of their initial diameter during manufacture. The folding occurs in part because the non-apertured sleeves are unable to deform controllably to such a highly deformed state. The apertures of the present invention provide for controlled deformation of the stent-reduction sleeve during the compression process.

The compression process, sometimes referred to as crimping or roll-down, is used to reduce the profile of stents and allow ready insertion and positioning of the reduced stent within the delicate and sometimes tortuous vasculature within the body. The method applies to balloon-deployed stents and to self-expanding stents. For example, balloon-expandable stents are reduced in diameter and crimped onto an inflatable balloon where they retain their reduced diameter until deployed with the inflatable balloon. In another example, self-expanding stents are reduced in diameter and slipped into a delivery sheath that retains the compressed stent framework with a reduced diameter until the sheath is retracted during deployment of the stent in the body.

When deployed in the body, stent 120 provides support to vessel walls and effectively clears occlusions and other blockages in the region of deployment. To reduce the chance of restenosis or other medical conditions from occurring in the vicinity of the stent, stent 120 may include a drug-polymer coating 150 disposed on stent framework 122 of stent 120. As shown in FIGS. 2 through 7, a stent-reduction sleeve with suitable arrangements of apertures may be used to reduce or avoid the potential for cracking, flaking or abrasion of drug-polymer coating 150 during roll-down, and to reduce any non-uniform compression of crowns 126 and struts 128 of stent framework 122.

Stent 120 with or without drug-polymer coating 150 may be used, for example, as a cardiovascular stent, a peripheral stent, an abdominal aortic aneurysm stent, a cerebral stent, a carotid stent, or an endovascular stent. Stents 120 are designed and manufactured to accommodate a range of vessel diameters and length of blocked or occluded vessels. Insertion of stent 120 into a vessel of the body helps treat, for example, heart disease, various cardiovascular ailments, and other vascular conditions. Catheter-deployed stent 120 typically is used to treat one or more blockages, occlusions, stenoses, or diseased regions in the coronary artery, femoral artery, peripheral arteries, and other arteries in the body. Treatment of vascular conditions involves the prevention or correction of various ailments and deficiencies associated with the cardiovascular system, the cerebrovascular system, urinogenital systems, biliary conduits, abdominal passageways and other biological vessels within the body. Generally tubular in shape with flexibility to bend along a central axis, stent 120 expands with the help of an inflatable balloon 130 or self-expands when released for a self-expanding version of stent 120.

Catheter 110 of an exemplary embodiment of the present invention includes inflatable balloon 130 that expands and deploys stent 120 within a vessel of the body. Inflatable balloon 130 is coupled to stent-delivery catheter 110 between catheter body 112 and stent framework 122. Distally mounted inflatable balloon 130 is attached to catheter body 112, and then stent framework 122 is positioned over the balloon and compressed to crimp sleeve-compressed stent 120 onto inflatable balloon 130.

Stent 120 is coupled to catheter 110, and may be deployed by pressurizing inflatable balloon 130 coupled to the stent and expanding stent 120, thereby expanding inflatable balloon 130 to a prescribed diameter. A flexible guidewire (not shown) traversing through a guidewire lumen 114 inside catheter 110 helps guide stent 120 to a treatment site, and once stent 120 is positioned, balloon 130 is inflated by pressurizing a fluid such as a contrast fluid that flows through a tube inside catheter 110 and into balloon 130. Stent 120 is expanded by balloon 130 until a desired diameter is reached, and then the contrast fluid is depressurized or pumped out, separating balloon 130 from deployed stent 120. Alternatively, stent-delivery catheter 110 may include a delivery sheath (not shown) that retracts to allow expansion of and deploy a self-expanding version of stent 120. Before insertion into a blood vessel, stent 120 is reduced in diameter and inserted into the delivery sheath coupled to stent-delivery catheter 110.

The apertured stent-reduction sleeve of the present invention may be used with various stents and stent designs. In one example, stent framework 122 includes a plurality of stent framework rings 124, each stent framework ring 124 having a plurality of interconnected crowns 126 and struts 128. Stent framework rings 124 are sinusoidally shaped, continuously formed in a loop or ring with smooth, rounded corners at each bend that are referred to as crowns 126, and substantially straight segments in between crowns 126 that are referred to as struts 128. One or more crowns 126 on stent framework ring 124 connect to corresponding crowns 126 on adjacent stent framework ring 124. As stent 120 is deployed, crowns 126 and struts 128 bend and straighten as the stent is enlarged diametrically, with minimal contraction extensionally. Selected crowns 126 of stent framework ring 124 connect to corresponding crowns 126 on adjacent stent framework ring 124 with, for example, a welded joint. Alternatively, crowns 126 of stent framework ring 124 may connect to corresponding crowns 126 on adjacent stent framework ring 124 with a molded joint, such as when stent 120 is formed from polymeric materials by a molding or casting process. In one example, stent framework 122 is formed from crimped rings of small diameter wire, and connections between selected crowns 126 are made. In another example, stent framework rings 124 are initially cut from sheets of metal and bent in a framework ring formation tool, and then welded together at selected crowns 126. In another example, stent framework 122 is laser-cut or water-cut from a tube of metal. In yet another example, stent framework 122 is formed in a mold using a polymeric material and has molded joints. Crowns 126 of one stent framework ring 124 are connected to corresponding crowns 126 on adjacent stent framework ring 124.

Stent framework 122 may include a polymeric base or a metallic base such as stainless steel, nitinol, tantalum, MP35N alloy, platinum, titanium, a suitable biocompatible alloy, a suitable biocompatible material, or a combination thereof.

A variety of materials such as polyurethane and expandable polytetrafluoroethane may be used to form inflatable balloons 130 and delivery sheaths for self-expanding stents, as is known in the art.

Optionally, a drug-polymer coating 150 may be disposed on stent framework 122 to provide desired therapeutic properties. Exemplary drug-polymer coating 150 comprises one or more therapeutic agents 152 that are eluted with controlled time delivery after the deployment of stent 120 within the body. Therapeutic agent 152 is capable of producing a beneficial effect against one or more conditions including coronary restenosis, cardiovascular restenosis, angiographic restenosis, arteriosclerosis, hyperplasia, and other diseases or conditions.

Drug-polymer coating 150 includes, for example, therapeutic agent 152 such as rapamycin, a rapamycin derivative, a rapamycin analogue, an antirestenotic drug, an anti-cancer agent, an antisense agent, an antineoplastic agent, an antiproliferative agent, an antithrombogenic agent, an anticoagulant, an antiplatelet agent, an antibiotic, an anti-inflammatory agent, a steroid, a gene therapy agent, a therapeutic substance, an organic drug, a pharmaceutical compound, a recombinant DNA product, a recombinant RNA product, a collagen, a collagenic derivative, a protein, a protein analog, a saccharide, a saccharide derivative, a bioactive agent, a pharmaceutical drug, and combinations thereof.

Incorporation of a drug or other therapeutic agents 152 into drug-polymer coating 150 allows, for example, the rapid delivery of a pharmacologically active drug or bioactive agent within twenty-four hours following the deployment of stent 120, with a slower, steady delivery of a second bioactive agent over the next three to six months. The thickness of drug-polymer coating 150 may extend, for example, between 1.0 microns and 200 microns or greater in order to provide sufficient and satisfactory pharmacological benefit.

FIG. 2 illustrates a stent-reduction sleeve with an array of diamond-shaped apertures, in accordance with one embodiment of the present invention at 200. A stent-reduction sleeve 240 includes diamond-shaped apertures 242 array about the periphery of stent-reduction sleeve 240 that reduces and minimizes the folding of stent-reduction sleeve 240 during compression or roll-down of a stent framework 222. A stent 220 having a stent framework 222 is inserted into stent-reduction sleeve 240 prior to roll-down. Apertures 242 provide space for material comprising stent-reduction sleeve 240 to deform and bulge into as the sleeve and stent framework 222 are compressed.

In the exemplary stent shown in FIG. 2, stent framework 222 includes a plurality of stent framework rings 224, each stent framework ring 224 having a plurality of interconnected crowns 226 and struts 228. A portion of the crowns 226 of one stent framework ring 224 is connected to corresponding crowns of an adjacent stent framework ring 224. A central axis 216 of stent framework 222 is approximately aligned with a central axis of stent-reduction sleeve 240 prior to roll-down. A drug-polymer coating 250 may be disposed on stent framework 222 to provide desired therapeutic properties.

In one example, stent framework 222 has a slightly smaller outer diameter than stent-reduction sleeve 240, so that stent framework 222 is readily inserted into stent-reduction sleeve 240. The length of stent-reduction sleeve 240 is selected, for example, so that the ends of stent-reduction sleeve 240 extend beyond the ends of stent framework 222. As stent framework 222 and stent-reduction sleeve 240 are compressed with a stent-reducing tool or other suitable compression device, material comprising stent-reduction sleeve 240 deforms into apertures 242 and reduces the possibility of buckling or folding of stent-reduction sleeve 240.

FIG. 3 illustrates a stent-reduction sleeve with an array of longitudinally elongated slotted apertures, in accordance with one embodiment of the present invention at 300.

A stent-reduction sleeve 340 includes an array of longitudinally elongated slotted apertures 342 about the periphery of stent-reduction sleeve 340 to reduce and minimize the folding of stent-reduction sleeve 340 during compression or roll-down of a stent 320. A stent framework 322 of stent 320 is inserted into stent-reduction sleeve 340 prior to roll-down. Longitudinally elongated slotted apertures 342 provide space for material comprising stent-reduction sleeve 340 to deflect and deform as stent-reduction sleeve 340 and stent framework 322 are compressed. The elongated slots effectively form thin strips of sleeve material along a portion of the length of stent-reduction sleeve 340. As stent-reduction sleeve 340 is compressed radially, the thin strips of material are deflected inwardly as the widths of apertures 342 decrease.

In the exemplary stent shown in FIG. 3, stent 320 has stent framework 322 including a plurality of stent framework rings 324. Each stent framework ring 324 has a plurality of interconnected crowns 326 and struts 328. A portion of crowns 326 of one stent framework ring 324 is connected to corresponding crowns of adjacent stent framework ring 324. A central axis 316 of stent framework 322 is approximately aligned with a central axis of stent-reduction sleeve 340 prior to roll-down. A drug-polymer coating 350 may be disposed on stent framework 322 to provide desired therapeutic properties.

For example, stent framework 322 has a slightly smaller outer diameter than stent-reduction sleeve 340, so that stent framework 322 may be readily inserted into stent-reduction sleeve 340. The length of stent-reduction sleeve 340 is selected, for example, so that the ends of stent-reduction sleeve 340 extend beyond the ends of stent framework 322. As stent framework 322 and stent-reduction sleeve are compressed with a stent-reducing tool or other suitable compression device, material comprising stent-reduction sleeve 340 deflects into apertures 342 and reduces the possibility of buckling or folding of stent-reduction sleeve 340. The extended portions of stent-reduction sleeve 340 may fold or buckle, though are not load-bearing and any folding or buckling will be inconsequential.

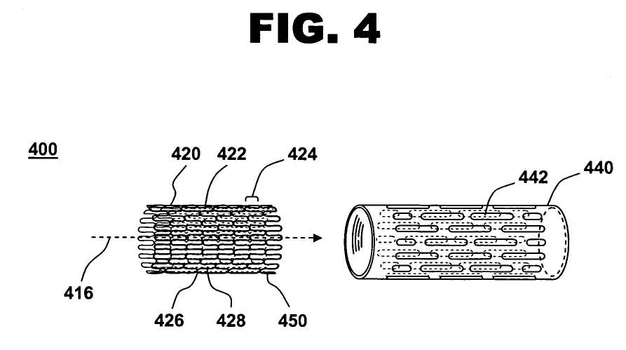

FIG. 4 illustrates a stent-reduction sleeve with an array of segmented longitudinal slotted apertures, in accordance with one embodiment of the present invention at 400.

A stent-reduction sleeve 440 includes an array of segmented longitudinal slotted apertures 442 about the periphery of stent-reduction sleeve 440 to reduce and minimize folding of stent-reduction sleeve 440 during compression or roll-down of a stent framework 422. A stent 420 of stent framework 422 is inserted into stent-reduction sleeve 440 prior to roll-down. Segmented longitudinal slotted apertures 442 provide space for material comprising stent-reduction sleeve 440 to deflect and deform into as stent-reduction sleeve 440 and stent framework 422 are compressed. The segmented elongated slots effectively form thin strips of sleeve material along a portion of the length of stent-reduction sleeve 440. As stent-reduction sleeve 440 is radially compressed, the thin strips of material are deflected inwardly as the widths of apertures 442 decrease.

In the exemplary stent shown in FIG. 4, stent 420 has a stent framework 422 including a plurality of stent framework rings 424, each stent framework ring 424 having a plurality of interconnected crowns 426 and struts 428. Selected crowns 426 of one stent framework ring 424 are connected to corresponding crowns of adjacent stent framework ring 424. When stent 420 is inserted into stent-reduction sleeve 440, a central axis 416 of stent framework 422 is approximately aligned with a central axis of stent-reduction sleeve 440 prior to roll-down. An optional drug-polymer coating 450 is disposed on stent framework 422 to provide desired therapeutic properties.

Stent framework 422 may have a slightly smaller outer diameter than the inside diameter of stent-reduction sleeve 440, so that stent framework 422 is readily inserted into stent-reduction sleeve 440. The length of stent-reduction sleeve 440 is selected, for example, so that the ends of stent-reduction sleeve 440 extend beyond the ends of stent framework 422. As stent framework 422 and stent-reduction sleeve are compressed with a stent-reducing tool or other suitable compression device, material comprising stent-reduction sleeve 440 deflects into apertures 442 and reduces the possibility of buckling or folding of stent-reduction sleeve 440. The extended portions of stent-reduction sleeve 440 may fold or buckle, though are not load-bearing and any folding or buckling will be inconsequential.

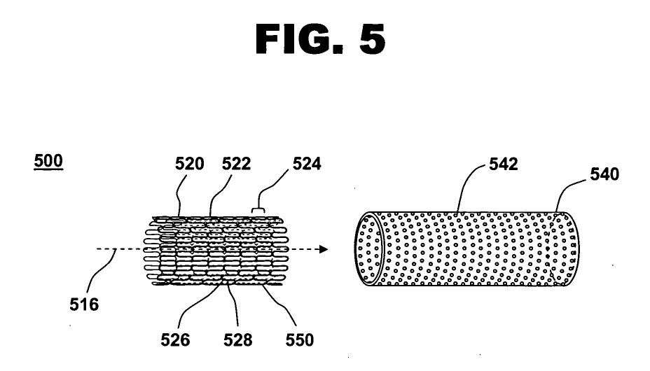

FIG. 5 illustrates a stent-reduction sleeve with a set of perforations, in accordance with one embodiment of the present invention at 500.

A stent-reduction sleeve 540 includes a plurality of perforated apertures 542 about the periphery of stent-reduction sleeve 540 to reduce and minimize the folding of stent-reduction sleeve 540 during compression or roll-down of stent framework 522. The set of perforations may be periodically positioned about stent-reduction sleeve 540. Perforated apertures 542 provide space for material comprising stent-reduction sleeve 540 to deform and bulge into as stent-reduction sleeve 540 and stent framework 522 are compressed. Perforated apertures 542 are small with respect to the width of stent framework rings 524. In one example, perforated apertures 542 have a diameter approximately equal to the thickness of stent-reduction sleeve 540. As stent-reduction sleeve 540 is radially compressed, material of stent-reduction sleeve 540 deforms into perforated apertures 542.

In the exemplary stent shown in FIG. 5, a stent 520 has a stent framework 522 including a plurality of stent framework rings 524 with a plurality of interconnected crowns 526 and struts 528. When inserted into stent-reduction sleeve 540, a central axis 516 of stent framework 522 is approximately aligned with a central axis of stent-reduction sleeve 540 prior to roll-down. A drug-polymer coating 550 may be disposed on stent framework 522 to provide desired therapeutic properties.

Stent framework 522 may have a slightly smaller outer diameter than the inside diameter of stent-reduction sleeve 540, so that stent framework 522 is readily inserted into stent-reduction sleeve 540. The length of stent-reduction sleeve 540 is selected, for example, so that the ends of stent-reduction sleeve 540 are coincident with or extend beyond the ends of stent framework 522. As stent framework 522 and stent-reduction sleeve are compressed with a stent-reducing tool or other suitable compression device, material comprising stent-reduction sleeve 540 deflects into perforated apertures 542 and reduces the possibility of buckling or folding of stent-reduction sleeve 540.

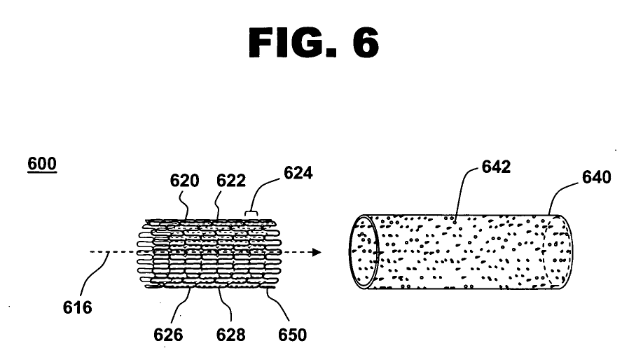

FIG. 6 illustrates a stent-reduction sleeve comprising a microporous material, in accordance with one embodiment of the present invention at 600.

A stent-reduction sleeve 640 is formed from a microporous material, the micropores serving as a plurality of microporous apertures 642. The micropores provide voids to reduce and minimize folding of stent-reduction sleeve 640 during compression or roll-down of a stent framework 622. Microporous apertures 642 provide voids for the microporous material to deform and bulge into as stent-reduction sleeve 640 and stent framework 622 are compressed. As stent-reduction sleeve 640 is radially compressed, material of stent-reduction sleeve 640 deforms into the microporous apertures 642.

In the exemplary stent shown in FIG. 6, a stent 620 has a stent framework 622 including a plurality of stent framework rings 624 having a plurality of interconnected crowns 626 and struts 628. Selected crowns 626 of one stent framework ring 624 are connected to corresponding crowns of an adjacent stent framework ring 624. An optional drug-polymer coating 650 may be disposed on stent framework 622 to provide desired therapeutic properties.

When stent 620 inserted into stent-reduction sleeve 640, a central axis 616 of stent framework 622 is approximately aligned with a central axis of stent-reduction sleeve 640 prior to roll-down. Stent framework 622 may have a slightly smaller outer diameter than the inside diameter of stent-reduction sleeve 640, so that stent framework 622 is readily inserted into stent-reduction sleeve 640. The length of stent-reduction sleeve 640 is selected, for example, so that the ends of stent-reduction sleeve 640 are coincident with or extend beyond the ends of stent framework 622. As stent framework 622 and stent-reduction sleeve 640 are compressed with a stent-reducing tool or other suitable compression device, material comprising stent-reduction sleeve 640 deflects into microporous apertures 642 and reduces the possibility of buckling or folding of stent-reduction sleeve 640.

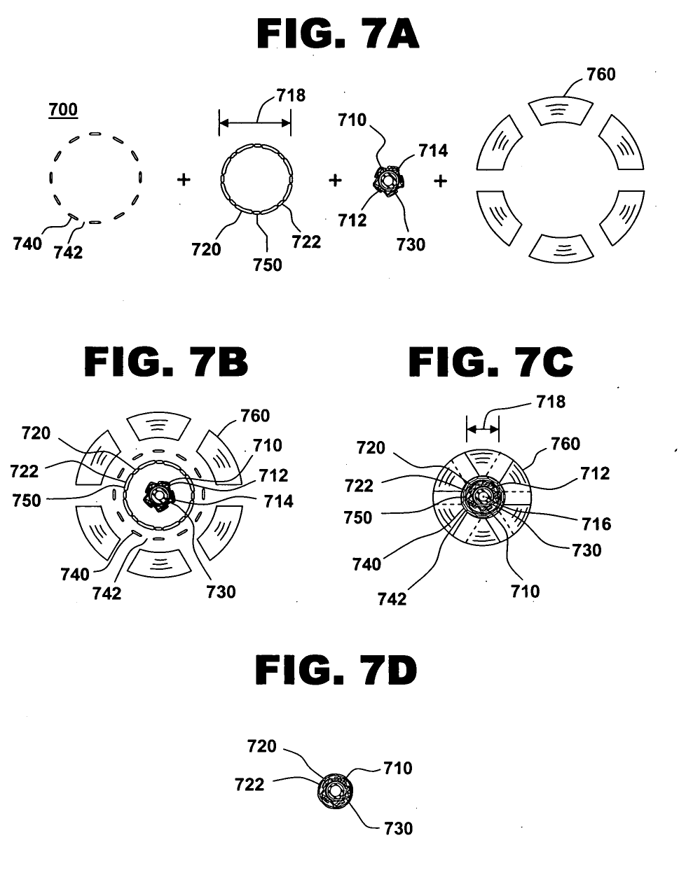

FIG. 7A, FIG. 7B, FIG. 7C and FIG. 7D show cross-sectional views of a method for reducing a stent, in accordance with one embodiment of the present invention at 700.

Illustrated in the cross-sectional view of FIG. 7A are a stent-reduction sleeve 740, a stent 720, an inflatable balloon 730 on a stent-delivery catheter 710, and a stent-reducing tool 760. Stent-reduction sleeve 740 has a plurality of apertures 742. Stent 720 includes a stent framework 722 having an optional drug-polymer coating 750 disposed on stent framework 722. Inflatable balloon 730 is shown folded and is coupled to a catheter body 712 of stent-delivery catheter 710. A hollow guidewire tube having a guidewire lumen 714 is positioned within a central lumen of catheter body 712. Stent-reducing tool 760 has a plurality of segmented jaws with arcuate inner surfaces for applying inwardly directed radial forces onto stent-reduction sleeve 740 and stent framework 722. Stent-reduction sleeve 740, stent 720, and inflatable balloon 730 coupled to catheter body 712 of catheter 710 are ready to be inserted into stent-reducing tool 760.

As illustrated in the cross-sectional view of FIG. 7B, stent 720 is inserted into stent-reduction sleeve 740, a thin-walled tube of a compressible material such as polytetrafluoroethylene, a nylon, polyurethane, a biocompatible polymer, a compressible polymer, or other materials that can compress stent 720 without causing excessive abrasions or otherwise damage stent 720 or any coatings such as a polymer-drug coating 750 disposed thereon. The thickness of stent-reduction sleeve 740 is, in one example, on the order of 0.010 inches thick. Stent-reduction sleeve 740 with apertures 742 and stent framework 722 are inserted into stent-reducing tool 760. Inflatable balloon 730 coupled to a stent-delivery catheter is inserted into stent framework 722. Catheter body 712 has a central lumen, and a tube positioned inside the central lumen of catheter body 712 has a guidewire lumen 714. An inflation port between the central lumen of catheter body 712 and an interior region of inflatable balloon 730 allows transport of an inflation fluid into and out from the interior region of inflatable balloon 730 through an annular region formed between catheter body 712 and the guidewire tube. Once stent framework 722, stent-reduction sleeve 740 and inflatable balloon 730 coupled to stent-delivery catheter 710 are inserted into stent-reducing tool 760, stent-reducing tool 760 is ready to reduce the diameter of stent framework 722.

Stent 720 with stent framework 722 and optional drug-polymer coating 750 is reduced in diameter with stent-reducing tool 760, as illustrated in the cross-sectional view of FIG. 7C. The stent-reduction sleeve 740 with apertures 742 is compressed towards a central axis 716 of stent framework 722 when stent-reducing tool 760 is actuated. A substantially uniform inwardly directed force is generated on the inserted stent framework 722. The stent diameter 718 of stent framework 722 is reduced based on the amount of compression, and stent framework 722 is crimped onto inflatable balloon 730, which is coupled to catheter body 712 of stent-delivery catheter 710.

When stent framework 722 has been compressed and reduced in diameter, reduced stent 720 and the stent-reduction sleeve are removed from the stent-reducing tool, and the stent-reduction sleeve is discarded, as illustrated in the cross-sectional view of FIG. 7D. A small amount of recoil may occur in stent framework 722 when stent 720 is removed from the stent-reduction sleeve and the stent-reducing tool, due in part to elastic behavior of stent framework 722 when stent framework 722 is compressed, reduced in diameter, and withdrawn from the stent-reduction sleeve and the stent-reducing tool along with stent-delivery catheter 710 and inflatable balloon 730.

In another embodiment with a self-expanding stent, the stent framework may be reduced and a delivery sheath (not shown) coupled to the catheter body is placed over the reduced stent framework to restrain the stent framework from expanding until positioned in the body and the delivery sheath is extracted. In this embodiment, an inflatable balloon between the stent framework and the catheter body is not included.

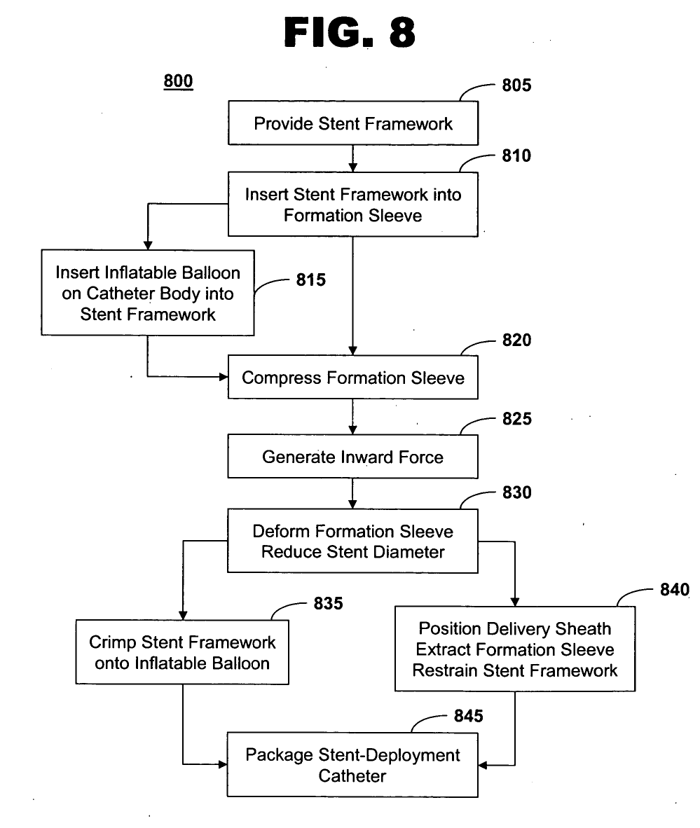

FIG. 8 is a flowchart of a method for reducing a stent, in accordance with one embodiment of the present invention at 800. Stent reduction method 800 includes various steps to reduce either a balloon-expandable stent or a self-expanding stent by using a stent-reduction sleeve.

A stent framework is provided, as seen at block 805. The stent is, for example, a cardiovascular stent, a peripheral stent, an abdominal aortic aneurysm stent, a cerebral stent, a carotid stent, or an endovascular stent. The stent includes a stent framework. In one example, the stent framework includes a plurality of stent framework rings, each stent framework ring having a plurality of interconnected crowns and struts. At least a portion of the crowns of one stent framework ring is connected to corresponding crowns of an adjacent stent framework ring with, for example, welded joints or molded joints. The stent framework rings are formed, for example, with a loop or ring of wire or a stamped-out ring pattern from a sheet of metal that is positioned into a framework ring forming tool and compressed to form the non-protruding crowns and protruding crowns with the desired pattern and size. In an alternative embodiment, the stent framework is formed from metal or polymers with a cast or a mold, the cast or mold having molded joints between connected crowns. In another embodiment, the stent framework is cut from small-diameter tubing with a laser or water-jet cutting tool.

The initial stent material may include, for example, a polymeric base or a metallic base such as stainless steel, nitinol, tantalum, MP35N alloy, platinum, titanium, a suitable biocompatible alloy, a suitable biocompatible material, or combinations thereof.

The stent framework is then cleaned using, for example, degreasers, solvents, surfactants, de-ionized water or other cleaners, as is known in the art.

A drug-polymer coating may be disposed on the stent framework. An exemplary drug polymer that includes a polymeric matrix and one or more therapeutic compounds is mixed with a suitable solvent to form a polymeric solution, and is applied using an application technique such as dipping, spraying, paint, or brushing. During the coating operation, the drug-polymer adheres to the stent framework and any excess drug-polymer solution may be removed, for example, by being blown off. In order to eliminate or remove any volatile components, the polymeric solution may be dried at room temperature or at elevated temperatures under dry nitrogen or another suitable environment. A second dipping and drying step may be used to increase the thickness of the drug-polymer coating, the thickness ranging between 1.0 microns and 200 microns or greater in order to provide sufficient and satisfactory pharmacological benefit.

The drug-polymer coating may be treated, for example, by heating the drug-polymer coating to a predetermined temperature, thereby driving off any remaining solvent or effecting any additional crosslinking or polymerization that is needed. The drug-polymer coating may be treated with air drying or low-temperature heating in an air, nitrogen, or other controlled environment. The coated or uncoated stent may be integrated into a system for treating vascular conditions such as heart disease by coupling the stent to the catheter.

The coated or uncoated stent framework is inserted into a stent-reduction sleeve, as seen at block 810. The stent-reduction sleeve includes a plurality of apertures formed therein. The plurality of apertures includes, for example, a lattice configuration of apertures, an array of diamond-shaped apertures, an array of longitudinally elongated slotted apertures, an array of segmented longitudinal slotted apertures, or a set of perforated apertures. The apertures may extend from end to end of the stent-reduction sleeve. Alternatively, a portion of the stent-reduction sleeve extending beyond an inserted stent framework may be void of apertures. The stent-reduction sleeve may comprise a thin-walled tube of a compressible material. The stent-reduction sleeve may comprise a material such as polytetrafluoroethylene, a nylon, polyurethane, a biocompatible polymer, a compressible polymer, or other material that allows compression of the inserted stent framework. Alternatively, the stent-reduction sleeve may comprise a microporous material such as porous Teflon™.

When a balloon-expandable stent is used, an inflatable balloon coupled to a stent-delivery catheter is inserted into the stent framework prior to compressing the stent-reduction sleeve, as seen at block 815. The inflatable balloon may be folded, and is positioned to extend beyond a distal end and a proximal end of the stent framework. In catheters with self-expandable stents, an inflatable balloon is not required. The stent-reduction sleeve with the inserted stent framework and the catheter body with or without an inflatable balloon is inserted into a stent-reduction tool to reduce the diameter of the stent framework therein.

The stent-reduction sleeve is compressed towards a central axis of the stent framework, as seen at block 820. The stent-reduction sleeve is compressed and reduced using a stent-reduction tool, which when activated, applies an inwardly directed radial force. In the example of a balloon-expandable stent, the stent framework is compressed plastically so that it largely retains its compressed or pre-deployment stent diameter when removed from the stent-reduction tool. In the example of a self-expanding stent such as a polymeric stent or a shape-memory alloy stent, the stent framework is compressed elastically so that it will return essentially to its uncompressed diameter when deployed unless restrained by the vessel walls or other constraint mechanism.

When the stent-reduction sleeve is compressed, a substantially uniform inwardly directed radial force is generated on the inserted stent framework, as seen at block 825. The stent-reduction sleeve cooperates with the stent-reduction tool to provide compression forces onto the stent framework, without abrading, scratching or otherwise damaging the stent framework and any drug-polymer coating formed thereon.

The stent-reduction sleeve is deformed in response to the compression, as seen at block 830. As the stent-reduction tool reduces the diameter of the inserted stent framework, the stent-reduction sleeve is also reduced in diameter. As the stent-reduction sleeve is reduced in diameter to sometimes as much as eighty percent of its pre-reduced diameter, the stent-reduction sleeve also undergoes significant diameter alterations. Apertures in the stent-reduction sleeve allow the reduction in diameter while minimizing or eliminating folding or buckling of the stent-reduction sleeve as the stent-reduction sleeve and stent framework are compressed. In cooperation with the stent-reduction sleeve and the apertures formed therein, a substantially uniform inwardly directed radial force is generated on the inserted stent framework. The inwardly directed radial force is uniformly applied to the stent framework by reducing or eliminating the folds, creases or buckling of the stent-reduction sleeve. The stent diameter of the stent framework is reduced based on the amount of the compressive force applied by the stent-reduction tool.

In the case of a balloon-expandable stent, the stent framework is crimped onto the inflatable balloon coupled to the stent-delivery catheter, as seen at block 835. Exemplary finished stents are reduced in diameter and formed, for example, with an interference fit that secures the stent onto the catheter.

In the case of a self-expandable stent, a delivery sheath is positioned over the compressed stent-reduction sleeve as the stent-reduction tool is disengaged from the stent framework, as seen at block 840. Prior to or after the positioning of the delivery sheath, the stent-reduction sleeve is extracted from between the inserted stent framework and the compressed stent-reduction sleeve. The self-expandable stent enlarges to contact the interior surface of the delivery sheath, and thereafter, the stent framework is restrained until deployed from the delivery sheath.

Radiopaque markers may be attached to the stent or catheter to aid in the placement of the stent within the body.

The stent-deployment catheter with the sleeve-compressed stent is packaged and stored until used, as seen at block 845. The catheter along with the drug-coated or non-coated stent may be sterilized and placed in a catheter package prior to shipping and storing. Additional sterilization using conventional medical means occurs before clinical use.

While the embodiments of the invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

1. A method of reducing a stent, the method comprising:
providing a stent framework;
inserting the stent framework into a stent-reduction sleeve, the stent-reduction sleeve including a plurality of apertures formed therein;
compressing the stent-reduction sleeve towards a central axis of the stent framework;
generating a substantially uniform inwardly directed radial force on the inserted stent framework; and
reducing a stent diameter of the stent framework based on the compression.

2. The method of claim 1 wherein the stent-reduction sleeve comprises a thin-walled tube of a compressible material.

3. The method of claim 1 wherein the stent-reduction sleeve comprises a material selected from the group consisting of polytetrafluoroethylene, a nylon, polyurethane, a biocompatible polymer, and a compressible polymer.

4. The method of claim 1 wherein the plurality of apertures are selected from the group consisting of a lattice configuration of apertures, an array of diamond-shaped apertures, an array of longitudinally elongated slotted apertures, an array of segmented longitudinal slotted apertures, and a set of perforated apertures.

5. The method of claim 1 wherein the stent-reduction sleeve comprises a microporous material.

6. The method of claim 1 wherein the stent includes a drug-polymer coating disposed on the stent framework.

7. The method of claim 1 further comprising:
deforming the stent-reduction sleeve responsive to the compression.

8. The method of claim 1 further comprising:
inserting an inflatable balloon coupled to a stent-delivery catheter into the stent framework prior to compressing the stent-reduction sleeve.

9. The method of claim 1 further comprising:
crimping the stent framework onto an inflatable balloon coupled to a stent-delivery catheter, wherein the inflatable balloon is inserted into the stent framework prior to compressing the stent-reduction sleeve.

10. The method of claim 1 further comprising:
positioning a delivery sheath over the compressed stent-reduction sleeve;
extracting the stent-reduction sleeve from between the inserted stent framework and the compressed stent-reduction sleeve; and
restraining the stent framework with the delivery sheath.

11. A system for treating a vascular condition, comprising:
a stent-delivery catheter including a catheter body; and
a sleeve-compressed stent coupled to the catheter body, the sleeve-compressed stent including a stent framework having a pre-deployment stent diameter that has been reduced by a stent-reduction sleeve, wherein the stent-reduction sleeve includes a plurality of apertures formed therein to minimize folding of the stent-reduction sleeve during a compression of the stent framework.

12. The system of claim 11 wherein the stent-delivery catheter includes an inflatable balloon used to expand the stent.

13. The system of claim 11 wherein the stent-delivery catheter includes a delivery sheath that retracts to allow expansion of the stent.

14. The system of claim 11 wherein the stent framework includes a plurality of stent framework rings, each stent framework ring having a plurality of interconnected crowns and struts; wherein at least a portion of the crowns of one stent framework ring is connected to corresponding crowns of an adjacent stent framework ring.

15. The system of claim 11 wherein the stent framework comprises one of a metallic base or a polymeric base.

16. The system of claim 15 wherein the metallic base is selected from the group consisting of stainless steel, nitinol, tantalum, MP35N alloy, platinum, titanium, a suitable biocompatible alloy, a suitable biocompatible material, and a combination thereof.

17. The system of claim 11 wherein the sleeve-compressed stent is crimped onto an inflatable balloon that is coupled to the stent-delivery catheter between the catheter body and the stent framework.

18. The system of claim 11 wherein the sleeve-compressed stent is selected from the group consisting of a cardiovascular stent, a peripheral stent, an abdominal aortic aneurysm stent, a cerebral stent, a carotid stent, and an endovascular stent.

19. The system of claim 11 further comprising:
a drug-polymer coating disposed on the stent framework.

20. A stent comprising:
a sleeve-compressed stent framework, the stent framework having a pre-deployment stent diameter that has been reduced by a stent-reduction sleeve, wherein the stent-reduction sleeve includes a plurality of apertures formed therein to minimize folding of the stent-reduction sleeve during a compression of the stent framework.

21. The stent of claim 20 wherein the sleeve-compressed stent framework includes a plurality of stent framework rings, each stent framework ring having a plurality of interconnected crowns and struts; wherein at least a portion of the crowns of one stent framework ring is connected to corresponding crowns of an adjacent stent framework ring.

22. The stent of claim 20 wherein the sleeve-compressed stent framework comprises one of a metallic base or a polymeric base.

23. The stent of claim 22 wherein the metallic base is selected from the group consisting of stainless steel, nitinol, tantalum, MP35N alloy, platinum, titanium, a suitable biocompatible, alloy, a suitable biocompatible material, and a combination thereof.

24. The stent of claim 20 wherein the sleeve-compressed stent is selected from the group consisting of a cardiovascular stent, a peripheral stent, an abdominal aortic aneurysm stent, a cerebral stent, a carotid stent, and an endovascular stent.

25. The stent of claim 20 further comprising:
a drug-polymer coating disposed on the stent framework.
